(19) United States
(12) Patent Application Publication
(10) Pub. No.: US 2025/0127696 A1
(43) Pub. Date: Apr. 24, 2025

(54) COMPOSITION FOR PREVENTING OR IMPROVING SKIN-AGING CONTAINING A THYMOL ESTER-BASED COMPOUND

(71) Applicant: AMOREPACIFIC CORPORATION, Seoul (KR)

(72) Inventors: Kyuhan KIM, Yongin-si (KR); Yeonju CHO, Yongin-si (KR); Suhyeon CHOI, Yongin-si (KR); Hyunjung CHOI, Yongin-si (KR)

(73) Assignee: AMOREPACIFIC CORPORATION, Seoul (KR)

(21) Appl. No.: 18/917,401

(22) Filed: Oct. 16, 2024

(30) Foreign Application Priority Data
Oct. 20, 2023 (KR) 10-2023-0141153

(51) Int. Cl. A61K 8/37; A61Q 19/08

(52) U.S. Cl. CPC A61K 8/37; A61Q 19/08; A61K 2800/92

(57) ABSTRACT

A composition capable of preventing or improving skin aging by controlling a senescence biomarker of melanocytes is disclosed. The composition includes a thymol ester-based compound as an effective ingredient, whereby when applied to a subject in which skin cells are flattened or enlarged, or β-galactosidase of melanocytes is activated, it can exhibit the effect of preventing or improving skin aging.

### CROSS REFERENCE TO RELATED APPLICATION

The present application claims priority to Korean Patent Application No. 10-2023-0141153 filed on Oct. 20, 2023, the entire contents of which is incorporated herein for all purposes by this reference.

### BACKGROUND OF THE INVENTION

### Field of the Invention

Disclosed herein is a composition for preventing or improving skin aging including a thymol ester-based compound, wherein the composition is capable of preventing or improving skin aging by controlling cellular senescence of melanocytes.

### Description of the Related Art

As life becomes more affluent, research is being conducted in various ways to delay the aging of the external parts as well as the internal parts of the body.

The area of greatest interest in the external aging is skin. The aging of the skin is divided into endogenous aging, which is a chronological aging that occurs over time according to biological processes, and photogenic aging, which occurs in a combination of degenerative changes and chronological aging in areas exposed to sunlight.

Due to these aging processes, the skin becomes dry, wrinkled, and loses elasticity. In particular, free radicals and reactive oxygen species, which are most importantly addressed in studies on skin aging, are known to cause oxidative stress to skin cells and inhibit or reduce the production of polymer substrates (collagen, hyaluronic acid, elastin) that maintain skin elasticity.

In this way, the skin aging can be promoted by the influence of various complex cells, but research on the correlation between melanocyte senescence and skin aging, the anti-aging mechanism of melanocytes, or the like is insufficient.

### SUMMARY OF THE INVENTION

### Technical Problem

In one aspect of the present disclosure, it is intended to provide a composition capable of preventing or improving skin aging by controlling cellular senescence of melanocytes.

### Technical Solution

In one aspect, the present disclosure provides a cosmetic composition for preventing or improving skin aging, which includes 3,4,5-trimethoxycinnamate thymol ester or a stereoisomer, salt, hydrate, or solvate thereof as an active ingredient, and reduces cellular senescence of melanocytes.

### Advantageous Effects

In one aspect, the composition according to the present disclosure can alleviate a change in which melanocytes are flattened or enlarged.

In another aspect, the composition according to the present disclosure can reduce the degree of β-galactosidase activation.

### BRIEF DESCRIPTION OF THE DRAWINGS

FIG. 1 shows the results of investigating drugs having a transcriptome pattern similar to a composition according to an embodiment of the present disclosure using RNA-seq.

FIG. 2 shows the results of measuring whether the predicted transcriptome age (RNA-age) of melanocytes decreases when treated with a composition according to an embodiment of the present disclosure.

FIG. 3 shows the results of observing a change in cell morphology when treated with a composition according to an embodiment of the present disclosure.

FIG. 4 shows the results of observing whether β-galactosidase in melanocytes decreases when treated with a composition according to an embodiment of the present disclosure.

### DETAILED DESCRIPTION OF THE INVENTION

Hereinafter, the present disclosure will be described in more detail by way of the following embodiments. However, the following embodiments are provided only for illustrative purposes to aid understanding of the present disclosure, and the scope and range of the present disclosure are not limited thereto.

In exemplary embodiments of the present disclosure, there is provided a composition for preventing or improving skin aging, which includes 3,4,5-trimethoxycinnamate thymol ester or a stereoisomer, salt, hydrate, or solvate thereof as an active ingredient, and reduces cellular senescence of melanocytes.

In another exemplary embodiment of the present disclosure, there is provided a method for preventing or improving skin aging, including administering a composition including 3,4,5-trimethoxycinnamate thymol ester or a stereoisomer, salt, hydrate, or solvate thereof as an active ingredient.

In one embodiment, the method for preventing or improving skin aging may be a method for preventing or improving skin aging by reducing a senescence biomarker of melanocytes.

In still another exemplary embodiment of the present disclosure, there is provided a use of 3,4,5-trimethoxycinnamate thymol ester or a stereoisomer, salt, hydrate, or solvate thereof for preparing a composition for preventing or improving skin aging.

In one embodiment, the composition for preventing or improving skin aging may be a composition for preventing or improving skin aging by reducing a senescence biomarker of melanocytes.

In yet another exemplary embodiment of the present disclosure, there is provided a non-therapeutic use of 3,4,5-trimethoxycinnamate thymol ester or a stereoisomer, salt, hydrate, or solvate thereof for preventing or improving skin aging.

In one embodiment, the non-therapeutic use may be for preventing or improving skin aging by reducing a senescence biomarker of melanocytes.

In yet another exemplary embodiment of the present disclosure, there is provided 3,4,5-trimethoxycinnamate thymol ester or a stereoisomer, salt, hydrate, or solvate thereof for preventing or improving skin aging.

In one embodiment, the 3,4,5-trimethoxycinnamate thymol ester or a stereoisomer, salt, hydrate, or solvate thereof may be used for preventing or improving skin aging by reducing a senescence biomarker of melanocytes.

In one embodiment, the 3,4,5-trimethoxycinnamate thymol ester is (5-methyl-2-propan-2-ylphenyl) (E)-3-(3,4,5-trimethoxyphenyl) prop-2-enoate, which may be represented by Chemical Formula 1 below:

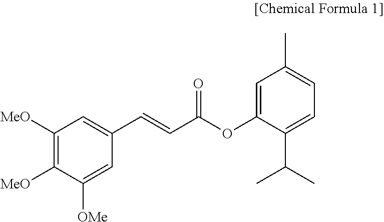 [SMILES: COc1cc(/C=C/C(=O)Oc2cc(C)ccc2C(C)C)cc(OC)c1OC]

As used herein, the term “isomer” includes in particular optical isomers (e.g., essentially pure enantiomers, essentially pure diastereomers or mixtures thereof), as well as conformation isomers (i.e., isomers which differ only in the angles of one or more chemical bonds), position isomers (in particular, tautomers) or geometric isomers (e.g., cis-trans isomers).

As used herein, the term “essentially pure”, for example, when used in connection with an enantiomer or diastereoisomer, means that a specific compound, for example an enantiomer or diastereoisomer, is present in an amount of at least about 90%, preferably at least about 95%, more preferably at least about 97% or at least about 98%, even more preferably at least about 99%, and even more preferably at least about 99.5% (w/w).

As used herein, the term “pharmaceutically acceptable” means that it can be or has been approved by the government or equivalent regulatory body as being suitable for use in animals, and more particularly, in humans, by avoiding significant toxic effects when used in conventional medicinal dosages, or is listed in a pharmacopoeia or recognized in other general pharmacopoeias.

As used herein, the term “pharmaceutically acceptable salt” means a salt according to one aspect of the present disclosure that is pharmaceutically acceptable and has the desired pharmacological activity of the parent compound. The salt may include (1) an acid addition salt formed of inorganic acid such as hydrochloric acid, hydrobromic acid, sulfuric acid, nitric acid, phosphoric acid, etc.; or formed of organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfate, gluconic acid, glutamic acid, hydroxynaphthoic acid, salicylic acid, stearic acid, muconic acid; or (2) a salt formed when an acidic proton present in the parent compound is substituted.

As used herein, the term “hydrate” refers to a compound to which water is bound, and is a broad concept that includes an inclusion compound with no chemical bonding force between water and the compound.

As used herein, the term “solvate” refers to a high-order compound formed between a molecule or ion of a solute and a molecule or ion of a solvent.

In one embodiment, the composition may reduce a cellular senescence biomarker of melanocytes.

In one embodiment, the cellular senescence biomarker may be β-galactosidase.

In one embodiment, the composition may be applied to a subject in which melanocytes are flattened or enlarged.

In one embodiment, the composition may be applied to a subject in which β-galactosidase of a melanocyte is activated.

In one embodiment, the composition may reduce an RNA age of melanocytes.

In one embodiment, the composition may be administered at a dosage of 0.001 mg/kg/day to 2000 mg/kg/day.

For example, the dosage may be 0.001 mg/kg/day or more, 0.01 mg/kg/day or more, 0.1 mg/kg/day or more, 1 mg/kg/day or more, 10 mg/kg/day or more, 100 mg/kg/day or more, 500 mg/kg/day or more, 1000 mg/kg/day or more, or 1500 mg/kg/day or more, and may be 2000 mg/kg/day or less, 1500 mg/kg/day or less, 1000 mg/kg/day or less, 500 mg/kg/day or less, 100 mg/kg/day or less, 10 mg/kg/day or less, 1 mg/kg/day or less, 0.1 mg/kg/day or less, or 0.01 mg/kg/day or less.

For example, the composition may be administered at a dosage of 0.5 mg/kg/day to 1500 mg/kg/day.

In one embodiment, the concentration of the 3,4,5-trimethoxycinnamate thymol ester or a stereoisomer, salt, hydrate or solvate thereof may be from 0.1 to 10 wt % based on the total weight of the composition.

For example, the concentration of the 3,4,5-trimethoxycinnamate thymol ester or a stereoisomer, salt, hydrate or solvate thereof may be 0.1 wt % or more, 0.5 wt % or more, 1 wt % or more, 3 wt % or more, 5 wt % or more, or 7 wt % or more, and 10 wt % or less, 7 wt % or less, 5 wt % or less, 3 wt % or less, or 1 wt % or less, or 0.5 wt % or less based on the total weight of the composition.

In one embodiment, the composition may be a cosmetic composition.

The external form of the cosmetic composition contains a cosmetically or dermatologically acceptable medium or base. It may be provided in any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule, or ionic (liposome) and nonionic vesicular dispersion, or in the form of a cream, a skin, a lotion, a powder, an ointment, a spray, or a concealer stick. These compositions can be prepared according to conventional methods in the art. The composition according to the present disclosure may also be used in the form of a foam or in the form of an aerosol composition further containing a compressed propellant.

The cosmetic composition according to one embodiment of the present disclosure is not particularly limited in its formulation, and may be formulated into cosmetics such as a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, and a body essence.

When the formulation of the cosmetic composition of the present disclosure is a paste, a cream, or a gel, animal fiber, plant fiber, wax, paraffin, starch, tragacanth, a cellulose derivative, polyethylene glycol, silicone, bentonite, silica, talc, or zinc oxide may be used as a carrier component.

When the formulation of the cosmetic composition of the present disclosure is a powder or spray, lactose, talc, silica, aluminum hydroxide, calcium silicate or polyamide powder may be used as a carrier component, and in particular, in the case of a spray, a propellant such as chlorofluorohydrocarbon, propane/butane or dimethyl ether may be additionally included.

When the formulation of the cosmetic composition of the present disclosure is a solution or an emulsion, a solvent, a solvating agent or an emulsifying agent is used as a carrier component, and the examples include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or a fatty acid ester of sorbitan.

When the formulation of the cosmetic composition of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier component.

When the formulation of the cosmetic composition of the present disclosure is a surfactant-containing cleansing, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, fatty alcohol, fatty acid glyceride, fatty acid diethanolamide, vegetable oil, linoline derivative, ethoxylated glycerol fatty acid ester, etc. may be used as a carrier ingredient.

The cosmetic composition of the present disclosure may further include a functional additive and a component included in a general cosmetic composition in addition to the above-described active ingredients. The functional additive may include a component selected from the group consisting of water-soluble vitamins, oil-soluble vitamins, polymer peptides, polymer polysaccharides, sphingo lipids, and seaweed extract.

In the cosmetic composition of the present disclosure, in addition to the above functional additive, a component included in a general cosmetic composition may be blended as necessary. In addition, other blending components, such as oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation promoters, cooling agents, anhydrotics, purified water, etc., may be additionally included.

In one embodiment, the composition may be a composition for skin external application.

The composition for skin external application is a generic term that may include anything that is applied externally to the skin, and may include various types of cosmetics, etc. In one embodiment, the composition may be a food composition.

The food composition according to the present disclosure may be a liquid or solid formulation, and may be a tablet, a capsule, a soft capsule, a pills, a granule, a beverage (drinking agent), a diet bar, a chocolate, a caramel formulation, or a confectionery formulation, but the formulation is not particularly limited.

In addition to the above active ingredients, the food composition according to the present disclosure may further include excipients, sugars, flavors, pigments, fats, proteins, etc., as needed.

Hereinafter, the present disclosure will be described in more detail by way of the following examples. However, the following examples are provided only for illustrative purposes to aid understanding of the present disclosure, and the scope and range of the present disclosure are not limited thereto.

### Examples

### Preparation of 3,4,5-trimethoxycinnamate thymol ester

Thymol trimethoxycinnamate thymol ester with CAS number 504394-57-4 (Melasolv) was obtained from COSMANN Co., Ltd. (Hwaseong-si, Gyeonggi-do, Korea).

### TEST EXAMPLE

### Test Example 1—Comparative Analysis of Transcriptome Patterns

RNA-seq was used to compare and analyze drugs having transcriptome patterns similar to RNA-seq data treated with 3,4,5-trimethoxycinnamate thymol ester (hereinafter, Melasolv).

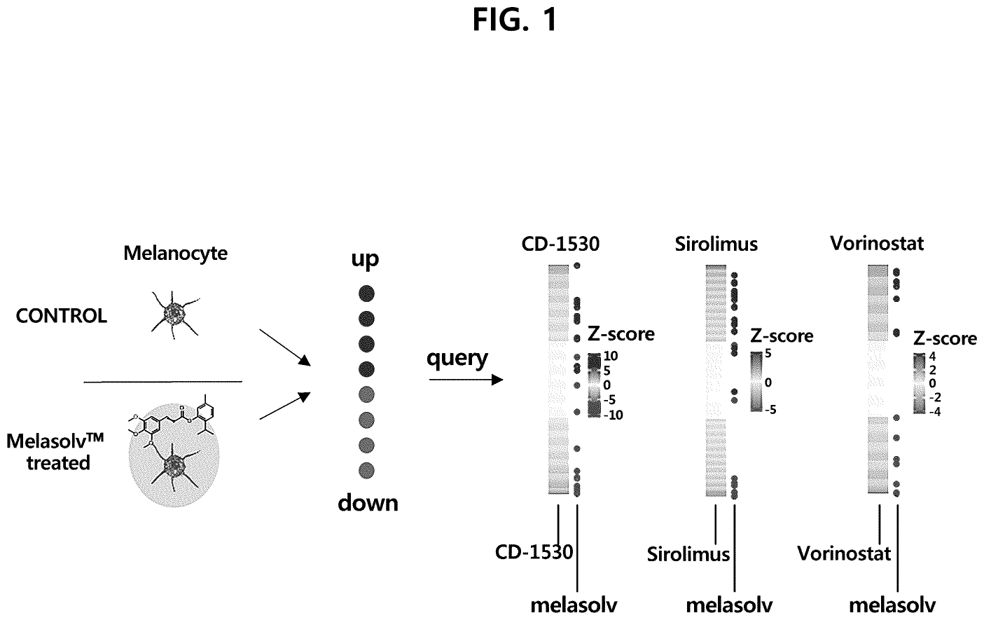

As a result of the comparative analysis, the analysis of the characteristics of the top 10 drugs having transcriptome patterns similar to Melasolv confirmed that they were highly related to anti-aging agents. In particular, as shown in FIG. 1, it was confirmed that the transcriptome patterns of CD-1530, Sirolimus, and Vorinostat, which are known to have excellent anti-aging effects, were similar to those of Melasolv.
The PubChem and CAS numbers of the substances used above are as follows: #CD 1530
PubChem CID: 9952709
CAS: 107430-66-0 #Sirolimus
PubChem CID: 5284616
CAS: 53123-88-9 #Vorinostat
PubChem CID: 5311
CAS: 149647-78-9

### Test Example 2—Observation of Predicted Transcriptome Age (RNA-Age)

Melanocytes were purchased from Cascade Biologics as cells cultured in the foreskin of newborn African-American males, and cultured using Medium254 medium mixed with human melanocyte growth supplement (Life technologies).

RNA was extracted from melanocytes treated with 10 ppm of Melasolv for 24 hours using TRIzol reagent (Life technologies). This RNA was used to prepare a library using the Illumina TruSeq Stranded Total RNA Library Prep kit (Illumina, San Diego, CA, USA), and then RNA-sequencing was performed using the NovaSeq 6000 platform (Illumina).

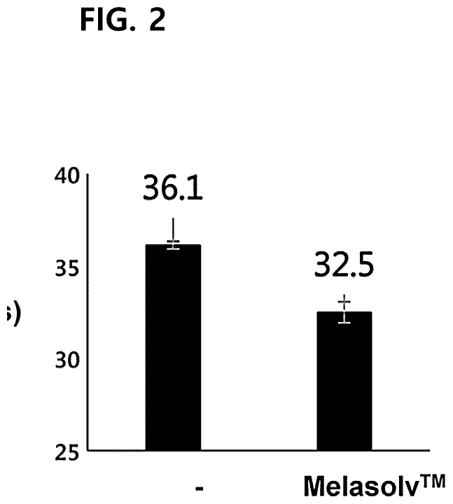

Thereafter, a model for predicting age based on approximately 16,000 gene expression values of RNA-seq data was applied to measure the RNA-age of the untreated control melanocytes (—in the graph of FIG. 2) and the RNA-age of the melanocytes treated with Melasolv. Specifically, the RNA-age used RNAAgeCalc package, which can calculate transcriptome age from RNAseq data. Among the transcriptome data, FPKM data was entered as input and skin tissue was entered as an argument for calculation.
(RNAAgeCalc pakage: https://bioconductor.org/packages/release/bioc/html/RNAAgeCalc.html,
Ren X, Kuan P F (2020). “A multi-tissue transcriptional age calculators.” PLOS ONE, 15(8),-20. doi: 10.1371/journal.pone.0237006)

As a result of observation, it was confirmed that the RNA-age of the melanocytes treated with Melasolv decreased by about 4 years, as shown in FIG. 2.

### Test Example 3—Observation of Cell Morphology

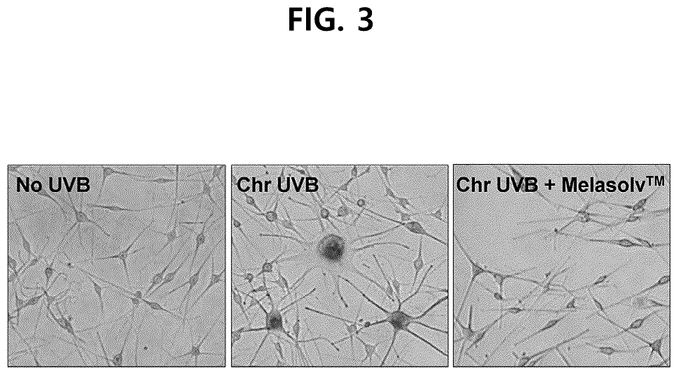

Melanocytes were irradiated with 5 mJ/cm² of UVB twice at 24-hour intervals using a 1 UVB UV irradiator (Bio-Sun), and then treated with Melasolv and incubated for 2 weeks. A photograph of the cells is taken using an optical microscope (ECLIPSE TS100 from NIKON), and the shape of the cells is examined. Referring to FIG. 3, when treated with ultraviolet (UVB) that induce cellular senescence (Chr UVB; Chronic UVB in FIG. 3), it could be confirmed that the skin cells were flattened or enlarged, as shown by three spots that were slightly larger than the others in the middle of the photograph. However, when additionally treated with Melasolv after UV treatment (UVB+Melasolv), no large spots were visible as in the case of UV treatment alone, and the appearance was similar to the control group that was not treated with UV, confirming that Melasolv reduces changes in skin cells caused by UVB.

### Test Example 4—Observation of β-Galactosidase, an Anti-Aging Indicator

Melanocytes were irradiated with 5 mJ/cm² of UVB twice at 24-hour intervals using a 1 UVB UV irradiator (Bio-Sun), and then treated with Melasolv and incubated for 2 weeks. To measure the degree of cellular senescence, a beta-galactosidase assay was performed using a beta-gal staining kit from Life technologies. A photograph was taken using an optical microscope (ECLIPSE TS100 from NIKON), and then the number of cells with stained beta-galactosidase was counted.

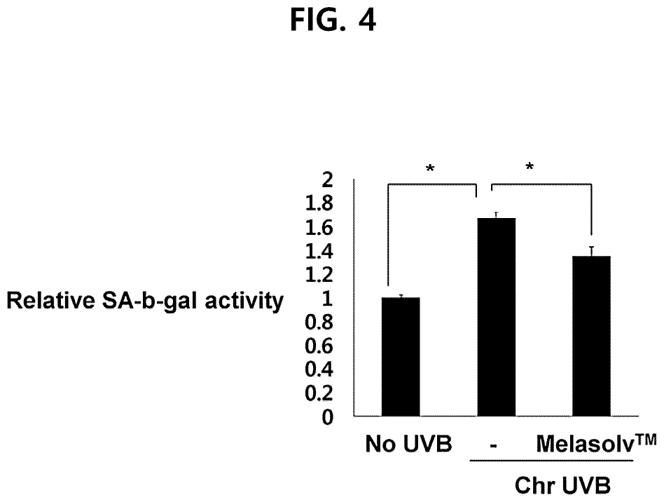

Referring to FIG. 4, it can be confirmed that when treated with Melasolv after irradiation with ultraviolet (UVB), β-galactosidase, an anti-aging indicator, was reduced to a statistically significant degree in melanocytes compared to the case where only ultraviolet rays were irradiated.

What is claimed is:

1. A method for preventing or improving skin aging, comprising:
administering an effective amount of a composition comprising 3,4,5-trimethoxycinnamate thymol ester or a stereoisomer, salt, hydrate, or solvate thereof as an active ingredient to a subject in need thereof,
thereby reducing cellular senescence of melanocytes.

2. The method according to claim 1, wherein the method reduces a cellular senescence biomarker of melanocytes.

3. The method according to claim 2, wherein the cellular senescence biomarker is β-galactosidase.

4. The method according to claim 1, wherein the composition is administered to a subject in which melanocytes are flattened or enlarged.

5. The method according to claim 1, wherein the composition is administered to a subject in which β-galactosidase of a melanocyte is activated.

6. The method according to claim 1, wherein the method reduces the RNA age of melanocytes.

7. The method according to claim 1, wherein the 3,4,5-trimethoxycinnamate thymol ester or the stereoisomer, salt, hydrate, or solvate thereof is administered at a dosage of 0.1 to 2000 mg/kg/day.

8. The method according to claim 1, wherein the concentration of the 3,4,5-trimethoxycinnamate thymol ester or the stereoisomer, salt, hydrate, or solvate thereof is 0.1 to 10 wt % based on the total weight of the composition.

9. The method according to claim 1, wherein the composition is a cosmetic composition.

10. The method according to claim 1, wherein the composition is a composition for skin external application.

11. The method according to claim 1, wherein the composition is a food composition.
